(19) United States
(12) Patent Application Publication
(10) Pub. No.: US 2003/0195511 A1
(43) Pub. Date: Oct. 16, 2003

(54) Device for and method of removing deleterious body tissue from a site within a patient

(75) Inventor: Robert Lawrence Barry, Kirkland, WA (US)

Correspondence Address: KNOBBE MARTENS OLSON & BEAR LLP, 2040 MAIN STREET, FOURTEENTH FLOOR, IRVINE, CA, 92614, US

(73) Assignee: Spiration, Inc.

(21) Appl. No.: 10/124,780

(22) Filed: Apr. 16, 2002

(51) Int. Cl. A61B 18/18

(52) U.S. Cl. 606/45

(57) ABSTRACT

A device, system, and method provides for removing deleterious tissue from healthy body tissue at a site inside a patient. The device includes a steerable electrosurgery device having a electrode to cut a core through tissue, including tissue surrounding a perimeter of the deleterious tissue, and has a lumen for aspirating the cored tissue from the patient. The electrode may form a closed loop, and may be the active electrode of an electrosurgery system. Further, the electrode may have a narrow profile. In addition, the electrode may be arranged to coagulate the cut tissue. The system includes the device and a seal to limit air leaks and bleeding resulting from the removal of cored tissue.

### BACKGROUND OF THE INVENTION

The present invention is generally directed toward a device for and method of removing deleterious body tissue from healthy body tissue at a site inside a patient. The present invention is more particularly directed toward a steerable electrosurgery device having a closed-loop electrode to cut a core through tissue, including tissue surrounding a perimeter of the deleterious tissue, and having a lumen for aspirating the cored tissue from the patient.

Cancer is a form of deleterious body tissue. Pulmonary cancer is the leading cause of cancer deaths in the United States. Early detection and proper treatment of cancerous tissue significantly improves survival rates.

Using traditional imaging means, asymptomatic, spherical, intrapulmonary lesions are found in about 1 of every 500 chest films. Solitary lesions having a diameter of 3 cm or less are presently defined as pulmonary nodules. Larger lesions are defined as masses. Currently, a pulmonary nodule proves to be a malignant tumor in about 40% of the cases, most often bronchogenic carcinoma but occasionally a solitary metastasis or carcinoid tumor.

A number of different procedures, techniques, and apparatus are available to treat pulmonary nodules, each having morbidity and mortality considerations that must be evaluated along with the operable risk to the patient. Any procedure involving the lungs is invasive and fraught with potential complications, including bleeding and lung air leaks. Lung tissue is very thin and fragile, and hence difficult to suture together without bleeding and air leaks. After a lung is resectioned, current procedures and techniques often restructure the remaining lung portion with suture staples.

Improved imaging techniques are providing an increased ability to detect intrapulmonary nodules early in their growth cycle. The improved techniques locate smaller intrapulmonary nodules than previous techniques. Nodules are now frequently discovered in the millimeter range. However, small pulmonary nodules are particularly difficult to locate and remove in surgery using existing devices and techniques. Because of trauma associated with the present techniques that require entering the chest cavity from the outside to remove a nodule, health care providers are reluctant to remove the small intrapulmonary nodules that are now being discovered until they know whether it is cancerous. The present procedure for small nodule management is to wait and see if the nodule grows rather than risking immediate surgery. If the small nodule is cancerous, it will grow over time. On confirmation of the growth, the nodule may be removed. However the survival rate will decrease because of the time delay.

In view of the foregoing, there in a need in the art for a new and improved apparatus and method of treating deleterious tissue located within healthy tissue, such as small intrapulmonary nodules, that minimizes potential complications and risks of other procedures. These risks typically include entering the lung cavity and removal of excessive tissue, and the associated risks of air leaks and bleeding. The present invention is directed to such an improved apparatus and method.

### SUMMARY OF THE INVENTION

The present invention provides a device for removing deleterious tissue from healthy body tissue at a site inside a patient. The device includes a steerable electrosurgery device having an electrode to cut a core through tissue, including tissue surrounding a perimeter of the deleterious tissue, and has a lumen for aspirating the cored tissue from the patient. The electrode may form a closed loop, may be substantially round, and may be the active electrode of an electrosurgery system. Further, the electrode may have a narrow profile. In addition, the electrode may be arranged to coagulate the cut tissue.

The invention further provides a system for removing deleterious tissue from healthy body tissue at a site inside a patient and sealing the remaining tissue. The system includes a steerable electrosurgery device having an electrode to cut a core through tissue, including tissue surrounding a perimeter of the deleterious tissue. The electrosurgery device also includes a lumen for aspirating the cored tissue from the patient. The system further includes a seal to limit air leaks and bleeding resulting from the removal of cored tissue. The seal may be deployable by the steerable electrosurgery device. The seal may include a plug inserted into the core resulting from the removal of cored tissue. The plug may be a glue, a solid member preferably expandable, or a sealing surface carried on an expandable structure. In an alternative embodiment, the seal includes a covering over the cored hole opening of the core resulting from the removal of cored tissue.

In accordance with a further embodiment of the present invention, a method is provided for removing deleterious tissue from healthy body tissue at a site inside a patient. The method includes the step of providing a steerable electrosurgery device having an electrode to cut a core through tissue, including tissue surrounding a perimeter of the deleterious tissue, and having a pathway for removing cored tissue from the patient. The method further includes the step of placing the electrode of steerable electrosurgery device inside the patient to a point where the electrode is in proximity with the deleterious tissue site. The method also includes the steps of coring tissue with the steerable electrosurgery device from the point in proximity to the deleterious tissue site to and including the deleterious tissue, and removing the cored tissue from the patient through the pathway. The method includes the step of sealing the core resulting from the removal of cored tissue from blood and air flow. The method may include the step of examining the cored tissue removed from the patient to confirm that all of the deleterious tissue has been removed. The electrode may form a generally round shape, may be the active electrode of an electrosurgery system, and may have a narrow profile. Furthermore, the electrode may be arranged to coagulate the cut tissue. The deployment of the seal may be by the steerable electrosurgery device. The seal may include a plug inserted into the core resulting from the removal of cored tissue. The plug may be glue, solid member preferably expandable, or a sealing surface carried on an expandable structure. In an alternative embodiment, the seal can include a covering over the cored hole opening of the core resulting from the removal of cored tissue.

In accordance with a further embodiment of the present invention, a device is provided for removing deleterious tissue from healthy body tissue at a site inside a patient. The device includes a steerable electrosurgery means for coring through tissue, including tissue surrounding a perimeter of the deleterious tissue, and an aspiration means for removing cored tissue from the patient.

In yet another embodiment of the present invention, a device is provided for biopsying deleterious tissue at a site inside a patient. The device includes a steerable electrosurgery device having an electrode to cut a core through tissue, including the deleterious tissue, and having a lumen for aspirating the cored tissue from the patient.

### BRIEF DESCRIPTION OF THE DRAWINGS

The features of the present invention which are believed to be novel are set forth with particularity in the appended claims. The invention, together with further objects and advantages thereof, may best be understood by making reference to the following description taken in conjunction with the accompanying drawings, in the several figures of which like referenced numerals identify identical elements, and wherein:

FIG. 1 is a simplified sectional view of a thorax illustrating a healthy respiratory system;

FIG. 2 illustrates a solitary pulmonary nodule in a lung;

FIG. 3 illustrates a monopolar electrosurgery system suitable for use with a steerable electrosurgery device in accordance with the present invention;

FIG. 4 is a perspective view, partially in section, of a closed-loop electrode carried on the distal end of a tubular member according to an embodiment of the invention;

FIG. 5 illustrates an initial step of placing the tubular member carrying the closed-loop electrode in the patient's air passageway;

FIG. 6 illustrates a step of cutting a core through the air passage wall into the lung parenchyma toward the intrapulmonary nodule;

FIG. 7 illustrates a further step of cutting a core through lung parenchyma tissue and toward the pulmonary nodule;

FIG. 8 is a partial section view illustrating the active electrode being advanced sufficiently to cut a core of tissue that includes the pulmonary nodule;

FIG. 9 illustrates the pulmonary nodule being aspirated from the patient and the tubular member withdrawn to the air passageway;

FIG. 10 illustrates a cross-section view of a bronchial branch with the core resulting from removal of cored tissue; and

FIG. 11 illustrates a cross-sectional view of a bronchial branch with a sealing surface covering the cored hole opening.

### DETAILED DESCRIPTION OF THE PREFERRED EMBODIMENTS

Briefly stated, the invention allows resection of a nodule by positioning a steerable electrosurgery device in an air passageway near the nodule, preferably using a steerable bronchoscope. The electrosurgery device is activated and steered toward the nodule. It cuts a core from the air passageway to and including the nodule. The nodule and other cored material are aspirated for biopsy, and the core is plugged.

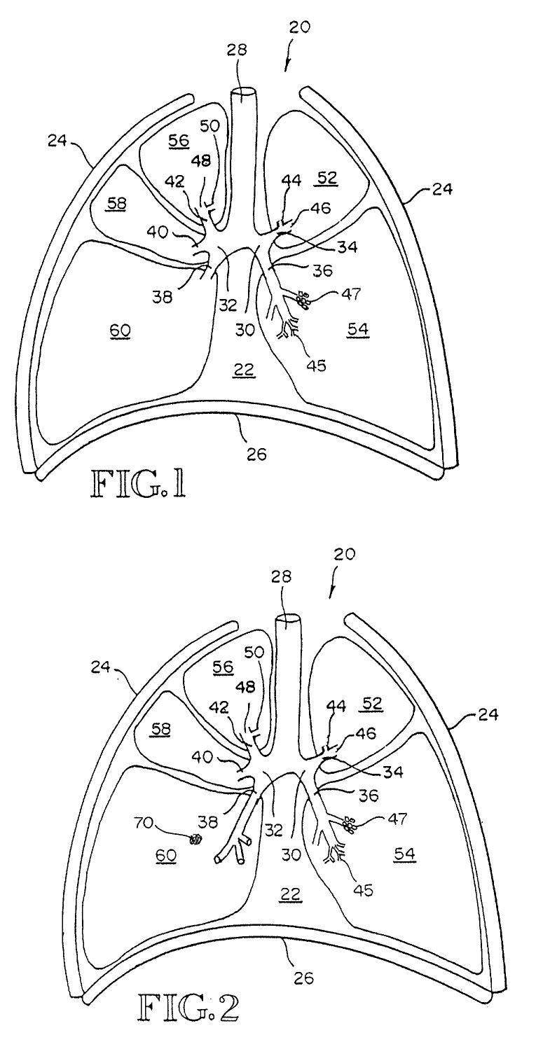

FIG. 1 is a sectional view of a healthy respiratory system. The respiratory system 20 resides within the thorax 22, which occupies a space defined by the chest wall 24 and the diaphragm 26.

The respiratory system 20 includes the trachea 28, the left mainstem bronchus 30, the right mainstem bronchus 32, the bronchial branches 34, 36, 38, 40, and 42 and sub-branches 44, 46, 48, and 50. The respiratory system 20 further includes left lung lobes 52 and 54 and right lung lobes 56, 58, and 60. Each bronchial branch and sub-branch communicates with a respective different portion of a lung lobe, either the entire lung lobe or a portion thereof. The sub-branches end in terminal bronchioles 45, and alveoli clusters 47.

Characteristic of a healthy respiratory system is the arched or inwardly arcuate diaphragm 26. As the individual inhales, the diaphragm 26 straightens to increase the volume of the thorax 22. This causes a negative pressure within the thorax. The negative pressure within the thorax in turn causes the lung lobes and alveoli to fill with air. When the individual exhales, the diaphragm returns to its original arched condition to decrease the volume of the thorax. The decreased volume of the thorax causes a positive pressure within the thorax, which in turn causes exhalation of the lung lobes.

FIG. 2 illustrates an intrapulmonary nodule in a portion of a lung. Aspects of the present invention are illustrated in this disclosure using a pulmonary nodule as an instance of deleterious body tissue. However, the present invention is also directed toward removing or biopsying other types of deleterious tissue, and is not limited to removing pulmonary nodules.

While intrapulmonary nodule 70 is illustrated in left lung lobe 54, pulmonary nodules may be located in any portion and in any structure of a lung. Pulmonary nodules are defined in current practice as lesions having a diameter of approximately 3 cm or less. They may have any contour (smooth, lobulated, or speculated) and may or may not be calcified. Larger lesions are called masses.

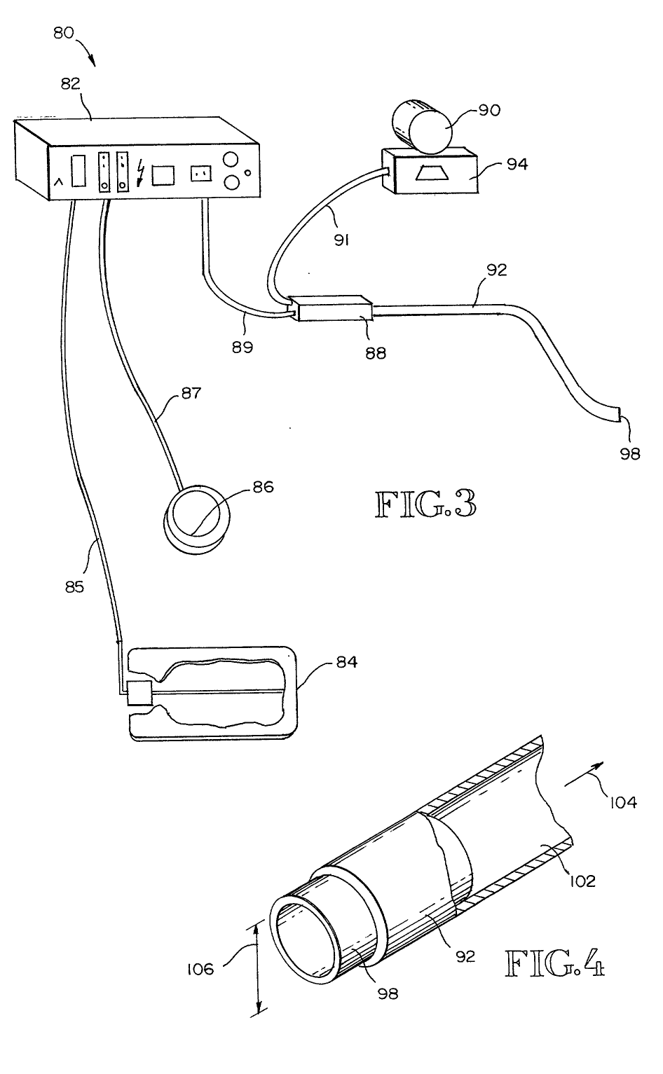

FIG. 3 illustrates a monopolar electrosurgery system suitable for use with a steerable electrosurgery device in accordance with the present invention. Electrosurgery system 80 includes an electrosurgery generator 82, a ground electrode 84, a ground electrode coupler 85, a foot control unit 86, a foot control unit coupler 87, a steerable electrosurgery device 88, an active electrode coupler 89, an aspiration pump 90, an aspiration coupler 91, a tubular member 92, a tissue receiver 94, and an active electrode 98. The depiction of a monopolar electrosurgery system in FIG. 3 is not intended to limit the practice of the present invention to only monopolar devices.

Electrosurgery generator 82 is coupled to ground electrode 84 by ground electrode coupler 85, to foot control unit 86 by foot control unit coupler 87, and to active electrode 98 by steerable electrosurgery device 88 and active electrode coupler 89. Tubular member 92 carries active electrode 98 and is a part of steerable electrosurgery device 88. Aspiration pump 90 is coupled to steerable electrosurgery device 88 and tubular member 92 by aspiration coupler 91. Cored tissue receiver 94 is coupled to aspiration coupler 91.

In operation, electrosurgery generator 82 supplies a source of electrical current typically in the radio frequency range to active electrode 98 and ground electrode 84 (which is sometimes known as a dispersive electrode). The current forms an electrical arc ahead of the active electrode 98 and volatizes the tissues, separating them as if they were cut. Ground electrode 84 provides a large surface for patient electrical contact, and is placed in electrical contact with the patient. Active electrode 98 directs the current flow into the tissue of the patient, and ground electrode 84 directs current flow from the patient to electrosurgery generator. The current waveform supplied by electrosurgery generator 82 may vary in strength and frequency, and it may be pulsed. The particular electrosurgery current waveform is selected to accomplish the objectives of the procedure being performed. The surgeon uses foot control unit 86 to control electrosurgery generator 82.

Tubular member 92 has a lumen for aspirating cored tissue from the patient and carries active electrode 98. Steerable electrosurgery device 88 is preferably configured to be carried in a steerable conduit or catheter, such as a bronchoscope with a steerable tip. In an alternative embodiment, the steerable electrosurgery device may be arranged to provide steering without being carried in a steerable conduit or catheter. The steps of removing deleterious tissue, such as nodule 70, according to an embodiment of the invention are described subsequently in conjunction with FIGS. 5-8. Cut tissue is aspirated from the patient through the lumen in tubular member 92 by aspiration pump 90 acting through aspiration coupler 91. Tissue receiver 94 receives tissue removed by aspiration.

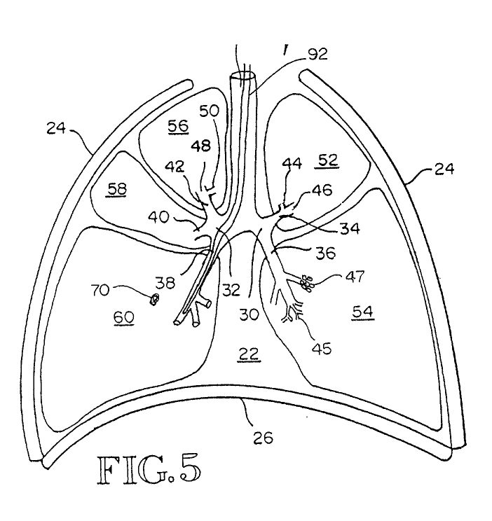

FIG. 4 is a perspective view, partially in section, of a closed-loop electrode carried on the distal end of a tubular member according to an embodiment of the invention. Active electrode 98 is carried on the end of tubular member 92 that is placed into the patient, and is coupled to the electrosurgery generator by the active electrode coupler. Tubular member 94 includes lumen 102 providing a path for aspirating cut tissue from active electrode 98 in aspiration pathway 104 toward the tissue receiver 94.

In a preferred embodiment, active electrode 98 is a closed-loop electrode consisting of radio frequency surgical materials having any cross-section shape and having an internal diameter 106 suitable for cutting a core through tissue, including tissue surrounding a perimeter of a deleterious tissue such as a pulmonary nodule. In an alternative embodiment, active electrode 98 is a round stainless steel member. In a further alternative embodiment, active electrode 98 has a narrow profile, with the material having a thickness in the range of 0.005 to 0.010 of an inch. According to an embodiment of the invention, active electrode 98 forms a closed-loop electrode, however, the loop is not required to lie in a single plane. In a further alternative embodiment, active electrode 98 may be sharpened. The inside diameter 106 of active electrode 98 may vary depending on the nodule size being removed. The surgeon may select an inside diameter 106 that is slightly larger than the nodule to be removed, so that the nodule and healthy tissue immediately adjacent thereto will be cored in a single pass. In an alternative embodiment, the inside diameter 106 can be smaller than the nodule, and the nodule removed in several passes, or a biopsy of the nodule taken in a single pass.

In an alternative embodiment, active electrode 98 may be carried within the outer perimeter of tubular member 94 until needed, and then extended for use. At least a portion of tubular member 94 is flexible, preferably the end carrying active electrode 98. The flexibility allows tubular member 98 to be steered. In a preferred embodiment, at least a portion of tubular member 92 is, and other portions of steerable electrosurgery device 88 may be, carried in a steerable catheter. The steerable catheter may be further carried in a bronchoscope having a steerable tip or a second steerable catheter. In an alternative embodiment, steerable electrosurgery device 88 may provide steering for tubular member 98 without using a steerable bronchoscope or catheter.

FIG. 5 illustrates an initial step of placing the tubular member carrying the closed-loop electrode in the patient's air passageway. This step places the closed-loop electrode at a point in an air passageway in proximity to the intrapulmonary nodule to be removed according to an embodiment of the invention. Tubular member 92 has a proximal end oriented toward the surgeon and a distal end that is placed in the patient. The distal portion of the tubular member 92 is preferably formed of flexible material capable of being steered and supporting the active electrode. It may be preformed at its distal end with a bend to assist in feeding into a bronchial branch, such as bronchial branch 38. The preformed bend may also initially orient tubular member 92 toward the wall of a bronchial branch to direct the active electrode toward the intrapulmonary nodule.

In a preferred embodiment, tubular member 92 is introduced and steered into the trachea and air passageways of the patient using a bronchoscope with a steerable tip. The distal tip of tubular member 92 including the closed-loop electrode is steered to a point where the closed-loop electrode is in proximity to the site of the intrapulmonary nodule 70. Tubular member 92 may be navigated using imaging information from computerized tomography (CT), ultrasound, or other system that provides information about the spatial relationship between the closed-loop electrode of tubular member 92 and the pulmonary nodule. A guidewire may also be used.

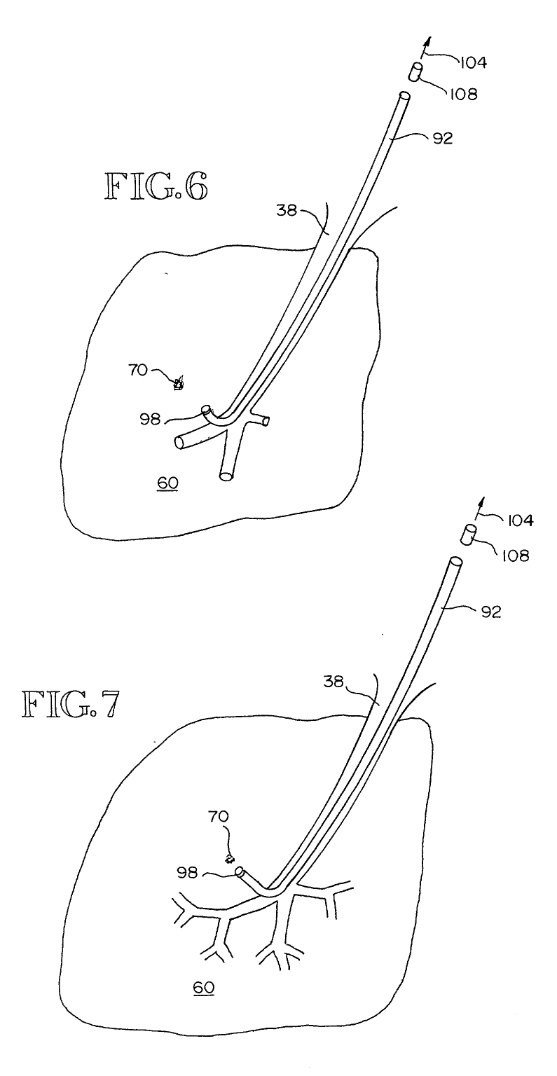

FIG. 6 illustrates a step of cutting a core through the air passage wall into the lung parenchyma toward the intrapulmonary nodule. In this step, the distal tip of tubular member 98 is orientated in direction of the pulmonary nodule, preferably using the steerable tip of the bronchoscope. The surgeon activates active electrode 98 by pressing the foot control unit of the electrosurgery system. This causes the electrosurgery generator to activate active electrode 98 by delivering a selected current waveform suitable for cutting a core of tissue. Tubular member 92 and the activated active electrode 98 of the steerable electrosurgery device are steered through the air passageway wall and toward the pulmonary nodule. This cuts a core through the air passageway wall and lung parenchyma tissue. The cored tissue 108 is removed from the patient through the lumen of tubular member 92 along aspiration pathway 104.

FIG. 7 illustrates a further step of cutting a core through lung parenchyma tissue and toward the pulmonary nodule. Tubular member 92 and the activated active electrode 98 of the steerable electrosurgery device continue to cut a core through the lung parenchyma tissue toward the pulmonary nodule 70. The tubular member 92 of the electrosurgery device is advanced toward pulmonary nodule 70. Movement is controlled with the steerable tip of the bronchoscope, and the cutting is controlled with the foot control unit. Progress toward the pulmonary nodule 70 is monitored by the imaging information and by examination of the cored tissue 108 aspirated from the patient.

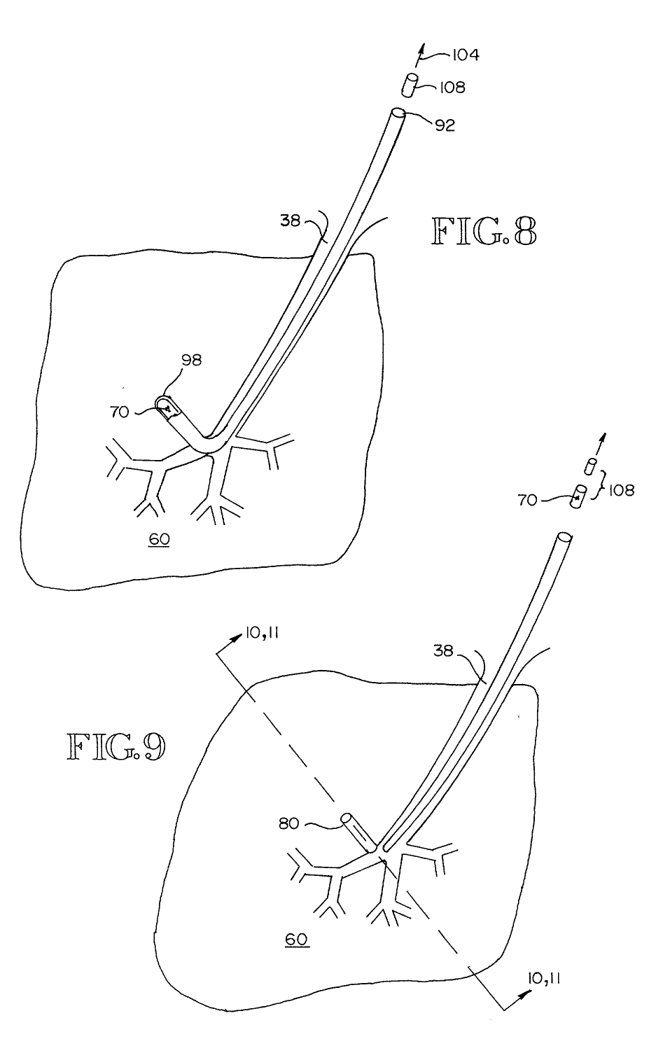

FIGS. 8 and 9 illustrate the active electrode having cored through tissue surrounding the pulmonary nodule, and confirmation being provided that the nodule has been removed from the patient. FIG. 8 is a partial section view illustrating the active electrode being advanced sufficiently to cut a core of tissue that includes the pulmonary nodule. Tubular member 92 and the activated active electrode 98 of the steerable electrosurgery device have advanced sufficiently to cut a core through the healthy tissue surrounding a perimeter of the pulmonary nodule 70. In a preferred embodiment, cutting a core through healthy tissue surrounding a perimeter of the pulmonary nodule 70 is desirable to provide an increased level of confidence that all deleterious tissue has been removed. In an alternative embodiment, an inside diameter of the active electrode 98 may be selected so that a core is cut through the pulmonary nodule 70, which could be used to biopsy a pulmonary nodule or a pulmonary mass.

FIG. 9 illustrates the pulmonary nodule being aspirated from the patient and the tubular member withdrawn to the air passageway. The cored tissue 108, including pulmonary nodule 70, is aspirated from the patient through the lumen in tubular member 92. Removal of pulmonary nodule 70 can be confirmed by visual or pathology examination of the aspirated cored tissue 108 before withdrawing tubular member 92 and the active electrode 98 from the former site of pulmonary nodule 70. Once the surgeon is satisfied, tubular member 92 is withdrawn to the air passageway. During withdrawal to the air passageway, the active electrode 98 may be activated and used for tissue coagulation of the core resulting from the removal of cored tissue 108. In this embodiment, the electrosurgery generator is configured to provide a current waveform suitable for tissue coagulation.

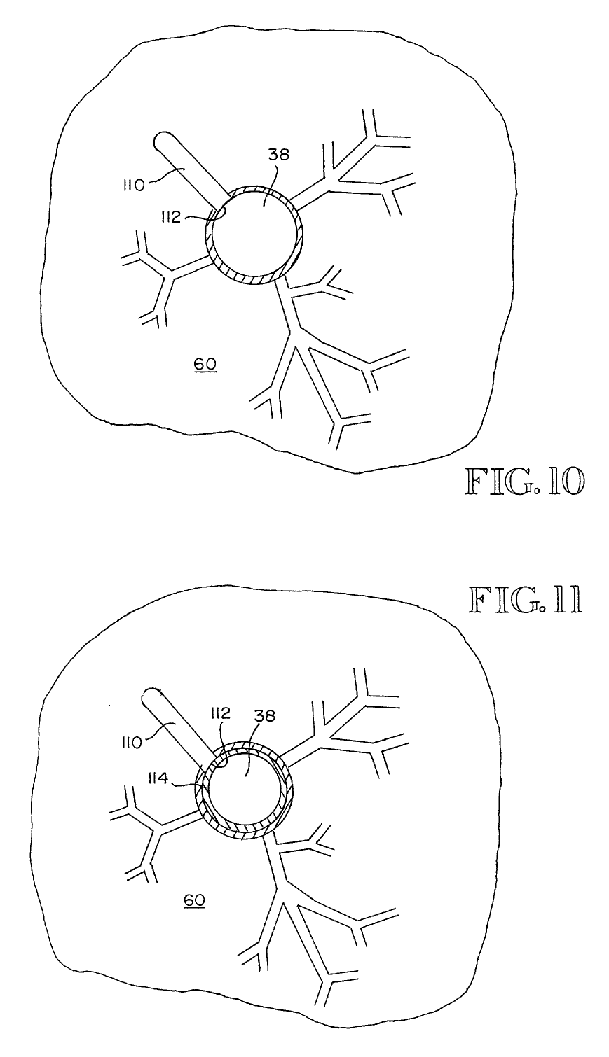

FIG. 10 illustrates a cross-section view of a bronchial branch with the core resulting from removal of cored tissue. An alternative embodiment of the invention provides for physically sealing the core resulting from removal of cored tissue 110 (hereafter “core 110”) to limit air leaks and bleeding resulting from the removal of cored tissue. Two seal locations may be used. A seal may be formed by placing a plug in the core 110, or by covering the cored hole opening 112 in the wall of the air passageway.

The plug is deployed into core 110 from the distal tip of tubular member 92 as it is removed from the core 110 to limit air leaks and bleeding from the core 110. In an alternative embodiment, the plug is deployed into core 110 from a catheter carried in tubular member 92. The plug may be comprised of a glue, a solid member (preferable expandable), or a sealing surface carried on an expandable structure such as a stent.

FIG. 11 illustrates a cross-sectional view of a bronchial branch with a sealing surface covering the cored hole opening. In this alternative embodiment, a sealing surface 114 covers and seals the cored hole opening 112 to limit air leaks and bleeding resulting from the removal of cored tissue. Sealing the cored hole opening 112 includes deploying a tubular member from the distal tip of tubular member 92. The tubular member has a peripheral sealing surface 114 that sealingly engages a portion of the air passageway wall around a periphery of the cored hole opening 112. The tubular member covers and seals the cored hole opening 112 while allowing air to pass. The tubular member includes a sealing membrane carried on a support member, which may be a stent. In an alternative embodiment, the tubular member may be an expandable tubular structure such as a silicone with a sealing surface.

In an alternative embodiment, sealing the cored hole opening 112 with sealing surface 114 includes deploying a plug from the distal tip of tubular member 92. The plug includes peripheral sealing surface 114, which is the peripheral surface of a plug that fills the entire cross-section of the air passageway, including the cored hole opening 112. The plug covers and seals the cored hole opening 112.

As can thus be seen from the foregoing, the present invention provides a new and improved device and method of treating pulmonary nodules that is less invasive and traumatic than present techniques. This is achieved using a steerable electrosurgery device that cores from an air passageway to the nodule, removes the nodule for biopsy, and plugs the core.

While particular embodiments of the present invention have been shown and described, modifications may be made, and it is therefore intended in the appended claims to cover all such changes and modifications which fall within the true spirit and scope of the invention.

What is claimed is:

1. A device for removing deleterious tissue from healthy body tissue at a site inside a patient, comprising:
a steerable electrosurgery device having an electrode to cut a core through tissue, including tissue surrounding a perimeter of the deleterious tissue, and having a lumen for aspirating the cored tissue from the patient.

2. The device of claim 1, wherein the electrode forms a closed-loop.

3. The device of claim 1, wherein the electrode is substantially round.

4. The device of claim 1, wherein the electrode is an active electrode of an electrosurgery system.

5. The device of claim 1, wherein the electrode has a narrow profile.

6. The device of claim 1, wherein the electrode is arranged to coagulate the cut tissue.

7. A system for removing deleterious tissue from healthy body tissue at a site inside a patient and sealing the remaining tissue, comprising:
a steerable electrosurgery device having a electrode to cut a core through tissue, including tissue surrounding a perimeter of the deleterious tissue, and having a lumen for aspirating the cored tissue from the patient; and
a seal to limit air leaks and bleeding resulting from the removal of cored tissue.

8. The system of claim 7, wherein the seal is deployable by the steerable electrosurgery device.

9. The system of claim 7, wherein the seal includes a plug inserted into the core resulting from the removal of cored tissue.

10. The system of claim 9, wherein the plug is selected from a group consisting of glue, solid material, solid expandable material, and a sealing surface carried on an expandable structure.

11. The system of claim 7, wherein the seal is a covering over the cored hole.

12. A method for removing deleterious tissue from healthy body tissue at a site inside a patient, including the steps of:
providing a steerable electrosurgery device having an electrode to cut a core through tissue, including tissue surrounding a perimeter of the deleterious tissue, and having a pathway for removing cored tissue from the patient;
placing the electrode of steerable electrosurgery device inside the patient to a point where the electrode is in proximity with the deleterious tissue site;
coring tissue with the steerable electrosurgery device from the point in proximity to the deleterious tissue site to and including the deleterious tissue;
removing the cored tissue from the patient through the pathway; and
sealing the core resulting from the removal of cored tissue from blood and air flow.

13. The method of claim 12, including the step of examining the cored tissue removed from the patient to confirm that all of the deleterious tissue has been removed.

14. The method of claim 12, wherein the electrode forms a closed loop.

15. The method of claim 12, wherein the electrode is substantially round.

16. The method of claim 12, wherein the electrode is an active electrode of an electrosurgery system.

17. The method of claim 12, wherein the electrode has a narrow profile.

18. The method of claim 12, wherein the electrode is arranged to coagulate the cut tissue.

19. The method of claim 12, wherein the seal is deployable by the steerable electrosurgery device.

20. The method of claim 12, wherein the seal includes a plug inserted into the core resulting from the removal of cored tissue.

21. The method of claim 20, wherein the plug is selected from a group consisting of glue, solid member preferably expandable, and a sealing surface carried on an expandable structure.

22. The method of claim 12, wherein the seal includes a covering placed over the cored hole opening of the core resulting from the removal of cored tissue.

23. A device for removing deleterious tissue from health body tissue at a site inside a patient, comprising:
steerable electrosurgery means for coring through tissue, including tissue surrounding a perimeter of the deleterious tissue; and
aspiration means for removing cored tissue from the patient.

24. A device for biopsying deleterious tissue at a site inside a patient, comprising:
a steerable electrosurgery device having a electrode to cut a core through tissue, including the deleterious tissue, and having a lumen for aspirating the cored tissue from the patient.
